(19) United States
(12) Patent Application Publication
(10) Pub. No.: US 2017/0128270 A1
(43) Pub. Date: May 11, 2017

(54) WOUND DRESSING

(71) Applicant: MEDTRADE PRODUCTS LIMITED, Crewe Cheshire (GB)

(72) Inventors: Andrew Hoggarth, Crewe Cheshire (GB); Craig Hardy, Cardigan (GB)

(73) Assignee: Medtrade Products Limited, Crewe Cheshire (GB)

(21) Appl. No.: 15/127,394

(22) PCT Filed: Mar. 19, 2015
(86) PCT No.: PCT/GB2015/050816

§ 371 Date: Sep. 19, 2016

(30) Foreign Application Priority Data
Mar. 19, 2014 (GB) 1404947.2

(51) Int. Cl. A61F 13/02

(52) U.S. Cl. CPC A61F 13/022; A61F 13/0209; A61F 13/025; A61F 13/0226; A61F 13/0289

(57) ABSTRACT

The present invention relates to a wound dressing composition for use as or in a wound dressing and to methods of making the wound dressing composition. The wound dressing composition comprises an absorbent material, an adhesive material and an anchor material attached to the absorbent material and the adhesive material. The anchor material is operable to maintain a link between the absorbent material and the adhesive material when the wound dressing composition is wet.

The present invention relates to a wound dressing composition for use as or in a wound dressing and to methods of making the wound dressing composition.

Topical wound dressings for use in the treatment of wounds or other openings at a physiological target site on a human or animal body which are exuding blood and/or other bodily fluids have been known for some time. The materials used to make the wound dressings act to absorb the blood and/or other bodily fluids, and also stem the flow of them from the body. Materials for wound dressings are described in, for example, WO2010031995 to MedTrade Products Limited, and are commercially available.

The management of exudate is of course essential and critical during wound care and surgical procedures. The aim of managing the exudate is essentially to provide a moist wound environment at the wound bed to minimise the risk of maceration, which in turn may reduce the negative impact upon the human or animal body and also shorten the length of time the patient will take to recover.

Wound dressings often comprise at least a quantity of an absorbent material. The purpose of the absorbent material is to absorb wound exudate from the wound, thereby drawing it away from the wound bed. This avoids the wound bed being overly wet which, as noted above, can be detrimental to the healing process.

When the absorbent material absorbs wound exudate, it typically expands and swells. In some instances, the absorbent material gels. This can result in a delamination of the absorbent material from other component parts of the wound dressing. This is undesirable, since it can for example result in absorbent material being left behind in the wound when the wound dressing is removed.

In order to address this problem, wound dressings comprising an absorbent material that is encapsulated within an encapsulating material have been prepared. In such wound dressings, the absorbent material remains intact within the encapsulating material during use, and the wound dressing maintains its structural integrity.

However, a problem with existing wound dressings comprising an absorbent material is that the dressing needs to encapsulate the absorbent pad within the dressing area such that on absorption of fluid it does not delaminate from the dressing structure. This can be achieved in a number of methods, examples include encapsulating the absorbent material within an encapsulating material such that the absorbent material is restricted to being smaller in size than the encapsulating material, and thus the wound dressing as a whole, or, incorporating an all over wound contact layer that encapsulates the absorbent pad area between the wound contact layer and the outer layer. In the two examples described, the absorbent material is in contact and potentially bonded, in a dry state, to the outer layer of the dressing. When the dressing absorbs fluid and is wetted, this bond breaks, hence the need for the encapsulating construction.

There therefore remains a need for a composition suitable for use as or in a wound dressing that can address the aforementioned problems. The present invention has been arrived at with the foregoing in mind.

According to the present invention, there is provided a wound dressing composition comprising an absorbent material, an adhesive material and an anchor material attached to the absorbent material and the adhesive material, whereby the anchor material is operable to maintain a link between the absorbent material and the adhesive material when the wound dressing composition is wet.

The wound dressing composition of the present invention does not delaminate when the composition is exposed to fluid, such as wound exudate. Thus, the wound dressing composition is beneficial in that wound exudate can be absorbed without the concern of the absorbent material separating from the wound dressing composition and being left behind in the wound. Further, the wound dressing composition of the present invention comprises an absorbent material that does not have to be encapsulated within an encapsulating material.

The term ‘wound’ is used herein to refer to any breach or opening in the skin or subcutaneous tissue at a physiological target site of a human or animal. Typically, the present invention relates to a physiological target site of a human. The term physiological target site may also be referred to herein as a wound site.

The term ‘wound dressing’ is used herein to refer to materials placed on a wound at a wound site that have absorbent, gelling, adhesive or protective properties. The wound dressings are not limited to a particular size or shape. The wound dressings may be placed in direct or indirect contact with the wound. The wound dressings may comprise a wound dressing composition as defined herein.

The term ‘absorbent material’ is used herein to refer to a physiologically acceptable material that is capable of absorbing fluid, such as wound exudate. The absorbent material referred to herein may be a superabsorbent material. Reference to an absorbent material also includes reference to a superabsorbent material unless expressed otherwise.

The term ‘superabsorbent material’ is used herein to refer to a hydrophilic material that is water-swellable, but not water soluble, and which is capable of absorbing fluid to greater than 2000%, preferably greater than 2500%, with a fluid retention of greater than 85%, preferably greater than 90%.

The term ‘water-swellable’ is used herein to refer to a material that, when contacted with water or water-containing fluid, will absorb the fluid and swell, but will not substantially dissolve in that fluid. In some instances, the material will gel upon contact with water or a water-containing fluid.

The term ‘water soluble’ is used herein to refer to a material that, when contacted with water or a water-containing fluid, will dissolve in that fluid.

The absorbent material may comprise a foam, such as a polymeric foam material, that is not a superabsorbent material. The polymeric foam may be polyurethane foam. However, the absorbent material is preferably a superabsorbent material.

The superabsorbent material may comprise a material selected from starch, cellulose and polymeric materials such as poly(vinyl alcohol) (PVA), poly(ethylene oxide) (PEO), and poly(acrylic acid). The poly(acrylic acid) may be a partially neutralised, lightly cross-linked poly(acrylic acid).

The superabsorbent material may be chemically modified. For example, the superabsorbent material may be a polymeric material obtained by graft polymerisation of acrylic acid onto the chain of carboxymethyl cellulose.

The terms “cross-linking” or “cross-linked” are used herein to refer to two or more polymer chains being linked by a primary bond, such as a covalent bond.

The term “lightly cross-linked” is used herein to refer to embodiments wherein the number of cross-linking primary bonds in the superabsorbent material is less than the total number of possible cross-linking bonds.

In some embodiments, the superabsorbent material is selected from polymeric materials such as PVA, PEO, and poly(acrylic acid), preferably a partially neutralised, lightly cross-linked poly(acrylic acid).

Alternative superabsorbent materials include carboxymethylcellulose, preferably in fibre form, and chitosan fibre derivatives. For example, the chitosan fibre derivatives may comprise the materials described in WO 2010/031995, the contents of which are incorporated herein by reference. Thus, the superabsorbent material may comprise a blend of chitosan fibres with a material that has an acid associated therewith, e.g. cellulose fibre coated with an acid such as acetic and/or lactic acid.

Typically, the superabsorbent material is a partially neutralised, lightly cross-linked poly(acrylic acid).

Typically, the superabsorbent material has an absorbance capacity of greater than 2000%. Preferably, the absorbance capacity is greater than 2500%.

Typically, the superabsorbent material has a fluid retention of greater than 85%. Preferably, the fluid retention is greater than 90%.

In one embodiment, the superabsorbent material is capable of absorbing fluid to greater than 2000% with a fluid retention of greater than 85%. In one embodiment, the superabsorbent material is capable of absorbing fluid to greater than 2500% with a fluid retention of greater than 90%.

The absorbent material may be in the form of fibres or a powder. Typically, the absorbent material is in the form of fibres. The fibres may be up to 100 mm in length, preferably from 5 to 75 mm, more preferably from 10 to 60 mm and most preferably from 30 to 55 mm. Good results have been observed using fibres in the range of 38 to 52 mm in length.

The absorbent material may be in the form of a woven or non-woven fibrous layer. Preferably, the absorbent material is in the form of a non-woven fibrous layer. Where the absorbent material is in the form of a layer, it may comprise a wound facing surface and non-wound facing surface.

The term “wound facing surface” is used herein to refer to a surface of a layer of material that, in use, faces toward the wound site. The term “non-wound facing surface” refers to a surface of a layer of material that, in use, faces away from the wound site.

The absorbent material may gel on contact with water or body fluid(s). The absorbent material may be a gelling or semi-gelling material.

The term ‘gelling material’ is used herein to refer to a material in which substantially all of the components therein may gel upon contact with water or body fluid(s). For example, it may comprise a fibrous material wherein substantially all of the fibres are capable of gelling upon contact with water or body fluid(s).

The term ‘semi-gelling’ is used herein to refer to a material that comprises a mixture of components, some of which gel upon contact with water or body fluid(s) and some of which do not. For example, a semi-gelling absorbent material may comprise a combination of fibres, some of which gel upon contact with water or body fluid(s) and some of which do not.

The term ‘non-gelling’ is used herein to refer to a material in which substantially all of the components therein do not gel upon contact with water or body fluid(s). For example, it may comprise a fibrous material wherein substantially all of the fibres are incapable of gelling upon contact with water or body fluid(s).

Typically, in wound dressings comprising an absorbent material that gels, the risk of delamination is increased. However, as described herein, the presence of the anchor material in the wound dressing composition prevents delamination. Beneficially, this provides for the use of a wound dressing composition that can incorporate an absorbent material that at least partially gels to be utilised without concern that some of it will be left behind at the wound site once the wound dressing composition is removed.

The adhesive material may consist of, or may comprise, any suitable physiologically acceptable adhesive.

The adhesive material may be a pressure sensitive adhesive, a heat-bonding adhesive, and the like. Typically, the adhesive material is a pressure sensitive adhesive. In some embodiments, the anchor material may be attached to the adhesive material by contacting the anchor material with the adhesive material and applying pressure.

The adhesive material may be polymeric. Typically, the adhesive material is selected from acrylic adhesives, polyurethane adhesives, and silicone adhesives.

The adhesive material may be in the form of a layer. The adhesive layer comprises a wound facing surface and a non-wound facing surface.

The adhesive material may cover the whole or a part of a non-wound facing surface of the anchor material. The adhesive material may cover around 50-100% of the non-wound facing surface of the anchor material, preferably from around 70-80% coverage and most preferably around 75% coverage. In some embodiments, the adhesive material may cover 100% of the non-wound facing surface of the anchor material. It is noted that, the higher the coverage of adhesive material, the stronger the bond between the adhesive material and the anchor material.

Where the adhesive material covers less than 100% of the non-wound facing surface of the anchor material, it may be located in intervals across such a surface. In such embodiments, the adhesive material may be located in regular or irregular intervals, preferably regular intervals.

The adhesive material may have a surface area greater than that of the anchor material and the absorbent material. In such embodiments, the adhesive material provides a border that outwardly extends beyond one or more edges of the anchor material and the absorbent material. Beneficially, the adhesive material provides a dual purpose of (a) providing an area of adhesive material to adhere to the anchor material and (b) providing a border around the adhered anchor material which can adhere to the skin surrounding the wound site. The adherence of the adhesive material to the skin of a patient can hold the wound dressing composition in place during use.

The anchor material is operable to maintain a link between the absorbent material and the adhesive material when the wound dressing composition is wet.

The anchor material may consist of, or may comprise, a material that is not water soluble and/or water swellable. The anchor material may have a lower absorbency than the absorbent material. The anchor material may be non-absorbent or substantially non-absorbent. The anchor material may be non-gelling.

Typically, the anchor material is in the form of fibres. The fibres can be of any desired diameter or length and can be formed into a textile fabric, a pad or a film for use.

The anchor material may be polymeric. Typically, the anchor material comprises polyethylene. Preferably, the polyethylene is high-density polyethylene (HDPE).

In alternative embodiments, the anchor material may be a textile, for example cotton and/or viscose. In such embodiments, the textile material would need to be attached to the absorbent material by needle punching.

Typically, the anchor material is in the form of a film or net. The film or net may comprise woven and/or non-woven fibres. Typically, the film or net comprises a woven structure. Such a woven structure comprises apertures. The apertures provide breathability to the anchor material and generally need to be large enough to allow the passage of fluid vapour therethrough. The aperture size is typically greater than 0.2 mm², preferably from 0.5 mm to 1.0 mm. The anchor material may comprise from 10-100 apertures per cm², preferably from 10 to 50 apertures per cm², more preferably from 15 to 30 apertures per cm².

The breathability of the anchor material is preferably measured as grams per square meter (gsm) over 24 hours at 37° C. Typically, the breathability of the anchor material is greater than 1500 gsm/24 hrs/37° C.

The anchor material may be in the form of a layer. The anchor layer comprises a wound facing surface and a non-wound facing surface. The anchor material may be referred to herein as a scrim or a net.

The thickness of the anchor material is typically from 10 microns to 200 microns, preferably from 20 to 100 microns, more preferably from 30 to 70 microns.

The weight of the anchor material is typically from 10 to 100 gsm, preferably from 20 to 80 gsm and more preferably from 30 to 70 gsm.

Typically, the anchor material is attached to a non-wound facing surface of the absorbent material. The anchor material may cover the whole of the non-wound facing surface of the absorbent material. Alternatively, it may cover only a part of the non-wound facing surface. Preferably, the anchor material covers the whole of the non-wound facing surface.

The anchor material may be attached to the absorbent material by methods including heat-bonding, heat meltable adhesives and/or needle punching. Preferably, the absorbent material is heat-bonded to the anchor material.

It has been discovered that heat-bonding a fibrous woven anchor material to a non-woven fibrous layer of absorbent material is particularly advantageous in the present invention. Whilst not wishing to be bound by theory, it is thought that the advantageous properties arise from the bond created between the absorbent material and the anchor material. More specifically, it is thought that in the heat-bonding process, a proportion of the fibres toward the bonding surface of the anchor material bond to a part of the fibres of the absorbent material. Thus, the fibres of the anchor material do not bond to the entire length of a fibre in the absorbent material, but only to a section thereof. The fibres in the non-woven absorbent material are entangled, which remains the case when the absorbent material is heat-bonded to the anchor material. It is thought that, following heat-bonding, the fibres of the absorbent material lose their absorbency properties, such as swelling and/or gelling properties, in the section of the fibre that is bound to the anchor material. When the wound dressing composition gets wet, e.g. on contact with wound fluid, the bond between the absorbent material and the anchor material remains intact, whilst the remaining fibres, including the non-bound section of the fibres bound to the anchor material, in the absorbent material serve to absorb the wound fluid.

The absorbent material may alternatively be needle punched to the anchor material which can also provide the advantages of providing a connection that does not delaminate once the wound dressing is wet. Again, whilst not wishing to be bound by theory, it is thought that, in the needle punching process, fibres from the non-woven fibrous layer of absorbent material can interlink with the fibres from the anchor material. The fibres in the non-woven absorbent material are entangled, which remains the case when the absorbent material is needle punched to the anchor material. When the wound dressing composition gets wet, e.g. on contact with wound fluid, the bond between the absorbent material and the anchor material remains intact, whilst the remaining fibres that are not interlinked at the bonding surface in the absorbent material serve to absorb the wound fluid. Attachment by needle punching is generally not as strong as attachment by heat-bonding, and so heat-bonding is preferred.

The absorbent material may alternatively be attached to the anchor material using a heat meltable adhesive. This method can also provide a connection that does not delaminate once the wound dressing is wet. Again, whilst not wishing to be bound by theory, the heat meltable adhesive between the two materials can encapsulate at least portion of the fibres of both materials at the bonding surface. It is thought that, following adhesion, the fibres of the absorbent material lose their absorbency properties, such as swelling and/or gelling properties, in the section of the fibre that is encapsulated by the adhesive. When the wound dressing composition gets wet, e.g. on contact with wound fluid, the bond between the absorbent material and the anchor material remains intact, whilst the remaining fibres, including the non-bound section of the fibres bound to the anchor material, in the absorbent material serve to absorb the wound fluid.

In some embodiments of the present invention, there is provided a wound dressing composition comprising an absorbent material in the form of a non-woven fibrous layer heat-bonded to an anchor material in the form of a woven fibrous film.

The wound dressing composition may be multi-layered. For example, the wound dressing composition may be in the form of a trilayer composition, comprising an absorbent material in a first layer, an anchor material in a second layer attached to the first layer and an adhesive material in a third layer attached to the second layer. The multilayer wound dressing composition has been described herein as comprising first, second and third layers, although it may comprise further layers, such as fourth, fifth, sixth, seventh, eighth, ninth, tenth layers, or more. The further layers may comprise any of the features referred to herein in relation to the first, second or third layers.

The wound dressing composition may additionally comprise further components of a wound dressing, such as for example, a backing material, skin-contact adhesives and/or wound contact materials.

The backing may comprise medical grade sheet materials such as but not limited to polymer films, thin foams and fabrics e.g. polyurethane films, polyurethane foams, nonwoven fabrics, etc.

Suitable skin contact adhesives may include, but are not limited to, acrylate, silicone, or polyurethane based adhesives. They can be based on hydrogels and can be porous to moisture with a high moisture vapour transmission rate. They can be applied from water emulsions, solvents or using hot melt systems. The adhesives should have a good skin tack but give minimal skin trauma on removal. They can constitute 100% coverage of the backing, or a partial coverage thereof in the form of a pattern or mesh.

Suitable wound contact materials may include, but are not limited to, non-adherent layers which give very low or no adhesion to skin, wicking layers to speed-up the absorption of fluid, active carrier layers for delivery of a therapeutic material (such as a pharmaceutical, haemostat, antimicrobial, wound healing agent, or scar reducing agent) and adhesive layers to help in holding the dressing in place while potentially reducing trauma on removal. They can be based on a foam, polymer mesh, a fabric (e.g. nonwoven), and a hydrogel adhesive or partial adhesive coverings. The term “wound contact” is used herein to refer to a material that directly contacts the wound.

Typically, the wound dressing composition comprises a wound contact material attached to the wound facing surface of the absorbent material. The wound contact material preferably provides a dual purpose of acting as a wicking layer to speed-up the absorption of fluid, such as wound exudate, whilst also preventing the leaching of absorbent material into the wound.

In some embodiments, the ratio of absorbent material to wound contact material is from 1:1 to 1:20. The ratio of such materials may vary however, as it is dependent on the selection of wound contact material and its weight and thickness.

The wound contact material is preferably in the form of a foam. Typically, the wound contact material forms a layer. The wound contact layer comprises a wound facing surface and a non-wound facing surface. In some embodiments, the foam layer may have a thickness of from 0.5 mm to 7 mm, preferably from 1.5 mm to 3.0 mm.

Typically, the wound contact material is hydrophilic, such as a hydrophilic foam. The wound contact material may comprise, or consist of, a hydrophilic polymeric material. The term ‘hydrophilic’ is used herein to refer to a material that has an affinity with water. In the present invention, the hydrophilic wound contact material has an affinity for water, which enables it to readily absorb water containing wound fluid.

Typically, the wound contact material comprises, or consists of, polyurethane, polyethylene or a textile material. The polyurethane may be in the form or a foam or a perforated film. The polyethylene may be in the form of a foam. The textile material may be in the form of a net.

Preferably, the wound contact material comprises, or consists of, a polyurethane foam. Typically, the wound contact material consists of a polyurethane foam.

The wound contact material may cover the whole or a part of the wound facing surface of the absorbent material. Typically, the wound contact material covers the whole of the wound facing surface of the absorbent material.

As noted herein, the wound contact material preferably acts as a wicking layer. In use, the wound contact material will absorb wound fluid and thus draw it away from the wound bed. This has the advantageous effect of reducing the volume of fluid at the wound bed, thus avoiding a wound bed that is overly wet by creating a moisture level that is more conducive to wound healing. It is preferable to draw the wound fluid away from the wound contact material, as over saturation of the wound contact material results in an overly saturated wound bed. In the present invention, the wound contact material and the absorbent material can act in synergy, whereby the wound contact material initially readily absorbs fluid from the wound site and the absorbent material subsequently absorbs the fluid from the wound contact material. The superior retention properties of the absorbent material mean it can retain the wound fluid and keep it away from the wound bed. Beneficially, this can enhance the healing rate of the wound.

The wound contact material may be attached to the absorbent material by heat-bonding, a pressure sensitive adhesive, heat meltable adhesives, needle punching and the like. Typically, the wound contact material is attached to the absorbent material by an adhesive bonding layer. The adhesive bonding layer may attach the two materials by heat bonding or pressure bonding. Preferably, heat bonding is used. The adhesive bonding layer is preferably in powder form. The adhesive bonding layer may comprise, or consist of, a polymeric material selected from polycalprolactone, polyamides, polyesters, ethylene copolymers and combinations of any two or more thereof.

The wound dressing composition may comprise a backing material. The backing material is typically attached to the adhesive material. The backing material is typically in the form of a layer, and represents the outermost layer, or the layer furthest away from the skin, of the wound dressing composition.

Beneficially, the backing material can act as a barrier to prevent contamination of the wound by contaminants such as bacteria. The backing material may also be waterproof.

Typically, the backing material forms a layer. The backing layer may be attached to the adhesive material by any appropriate means known to a person skilled in the art. Where the adhesive material comprises, or consists of, a pressure sensitive adhesive, the backing material can simply be contacted with the adhesive material and appropriate pressure applied. The backing material is preferably in the form of a film, more preferably a non-woven film.

The backing material may comprise, or consist of, a polymeric material. Typically, the backing material comprises, or consists of, polyurethane, polyethylene, and polyester. The polyester may be non-woven. Preferably, the backing material is polyurethane.

The wound dressing composition may further comprise a skin protection layer.

The skin protection layer may provide an alternative means of adhering the wound dressing composition to the skin surrounding the wound site. In such embodiments, the skin protection layer may be attached to the wound facing surface of the adhesive material.

Preferably, the skin protection layer is attached to the wound facing surface of the border of the adhesive material, i.e. the part of the adhesive material that extends outwardly from the anchor material adhered thereto.

The skin protection layer contains an adhesive material for securing the dressing to the wound site, which may consist of a silicone material or pressure sensitive adhesive material. Silicone is ideally suited to application as the skin protection layer, since it can adhere to the skin in the same way as the adhesive material can, but it can be removed and reapplied with little irritation and damage. Also, the silicone material can be removed from the skin with reduced pain for the user compared to the adhesive material described herein.

The silicon layer may require a carrier material located between it and the adhesive material. The carrier material typically comprises a polymeric film. The carrier material may be the same material as the backing layer as described herein.

The skin protection layer adhered to the adhesive material may overlap with the non-wound facing surface of the anchor material. In such embodiments, the non-wound facing surface of the anchor material is attached to the adhesive material, as described herein, and is also attached to a part of the skin protection layer. This provides an increased stability for the wound dressing composition.

Alternatively, the skin protection layer may overlap with at least a part of the wound facing surface of the absorbent material or, if present, the wound facing surface of the wound contact material. Again, this provides increased stability to the wound dressing composition. It also provides a section of the skin protection layer that could be in direct contact with the wound site. Again, the gentle adherence of silicone material to the skin makes it ideally suited for contact with the wound site. Wounds and parts of wounds heal at different rates and stick to the wound dressing composition in different places and at different times. It is considered to be beneficial to have at least a part of the absorbent material, or the wound contact material if present, covered with a silicone material due to its gentle adherence to the wound site.

In some embodiments, the skin protection layer can extend across all or a part of the wound facing surface of the absorbent material or the wound contact material. In such embodiments, the skin protection layer is preferably perforated to facilitate absorption of the wound fluid by the wound contact material and/or the absorbent material whilst also providing a breathable composition and one that is gently adhered to the wound site.

According to a further aspect of the present invention, there is provided a wound dressing comprising a wound dressing composition as defined herein.

The wound dressing composition of the present invention, or a wound dressing comprising such wound dressing composition, may also comprise additional components mixed with any one or more of the material or layers described herein. Such additional components include, but are not limited to, pharmaceutical agents; wetting agents such as surfactants; growth factors; cytokines; agents which absorb agents which delay healing such as MMP's (matrix metalloproteinases) and elastase; and/or another wound dressing component, such as calcium, vitamin K, fibrinogen, thrombin, factor VII, factor VIII, clays such as kaolin, oxidised regenerated cellulose, gelatin, or collagen, etc.

Typical levels of any of these additional components could be from about 50 ppm up to about 50% by weight of the wound dressing composition. More typical levels would be less than 10%, still more typically less than about 5% by weight of the wound dressing composition. Additional components comprising less than about 1% by weight of the wound dressing composition are also envisaged by the present invention.

According to a further aspect of the present invention, there is provided a method of manufacturing a wound dressing composition as described herein, comprising the steps of:
(a) providing an absorbent material,
(b) attaching the absorbent material to a surface of an anchor material,
(c) attaching the opposing surface of the anchor material to an adhesive material, whereby the anchor material is operable to maintain the link between the absorbent material and the adhesive material when the wound composition is wet.

The absorbent material, adhesive material and anchor material are as defined herein.

The method of the present invention may comprise heat-bonding the absorbent material to the anchor material.

The material and/or components of the wound dressing composition described herein may be provided in a sterile or non-sterile form. Where the materials and/or components are initially provided in a sterile form, sterilisation may be carried out using any of the methods conventionally known in the art, such as gamma irradiation, electron beam treatment, heat treatment, x-ray, etc., or it may alternatively be carried out by treatment using ethylene oxide. Sterilisation using ethylene oxide is preferred. A material in a non-sterile form may be provided in combination with one or more preservatives. However, it is preferred that the wound dressing composition is provided in a pre-sterilised form.

The wound dressing composition of the present invention, or wound dressing comprising such a wound dressing composition, is typically sterilised prior to packaging using any of the methods described herein. This enables the physician or emergency responder to use the wound dressing composition or wound dressing directly from the packaging, thus saving time.

According to a further aspect of the present invention, there is provided a use of a wound dressing composition as defined herein, or a wound dressing as define herein, in absorbing fluid discharged from a physiological target, or in stemming a flow of a fluid discharged from a physiological target site.

According to a further aspect of the present invention, there is provided a wound dressing composition as defined herein, or a wound dressing as define herein, for use in absorbing fluid discharged from a physiological target, or for use in stemming a flow of a fluid discharged from a physiological target site.

Embodiments of the present invention will now be further described with reference to the following non-limiting examples and accompanying figures in which:

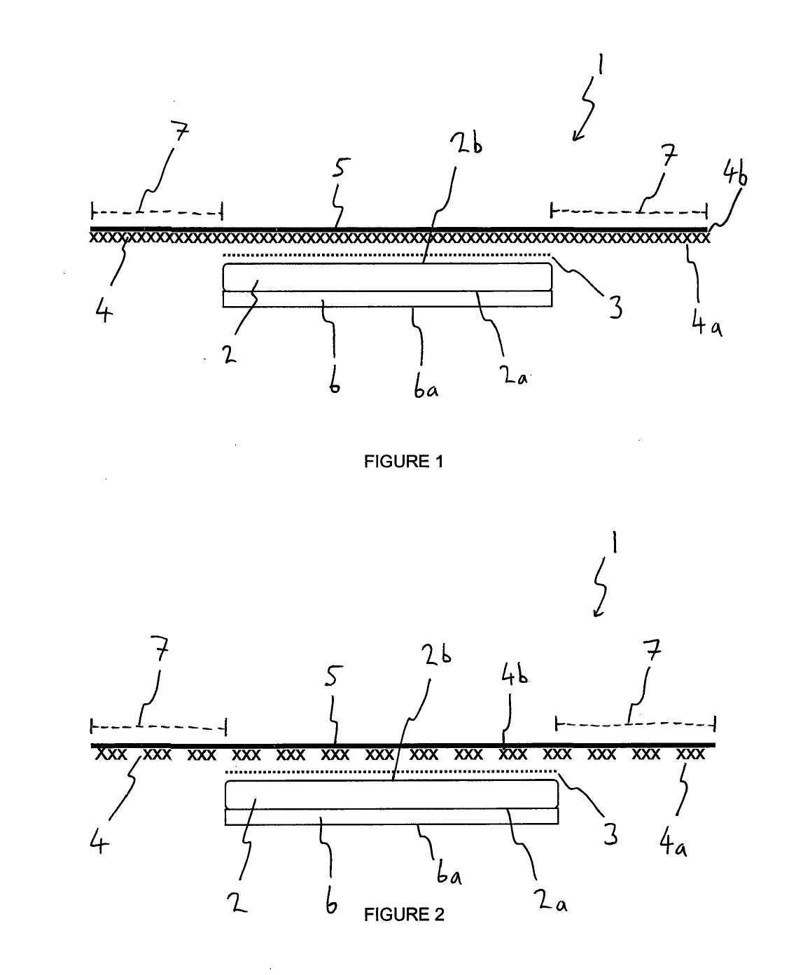

FIG. 1: is a cross-sectional representation of a wound dressing composition according to an embodiment of the present invention;

FIG. 2: is a cross-sectional representation of an alternative wound dressing composition of the present invention;

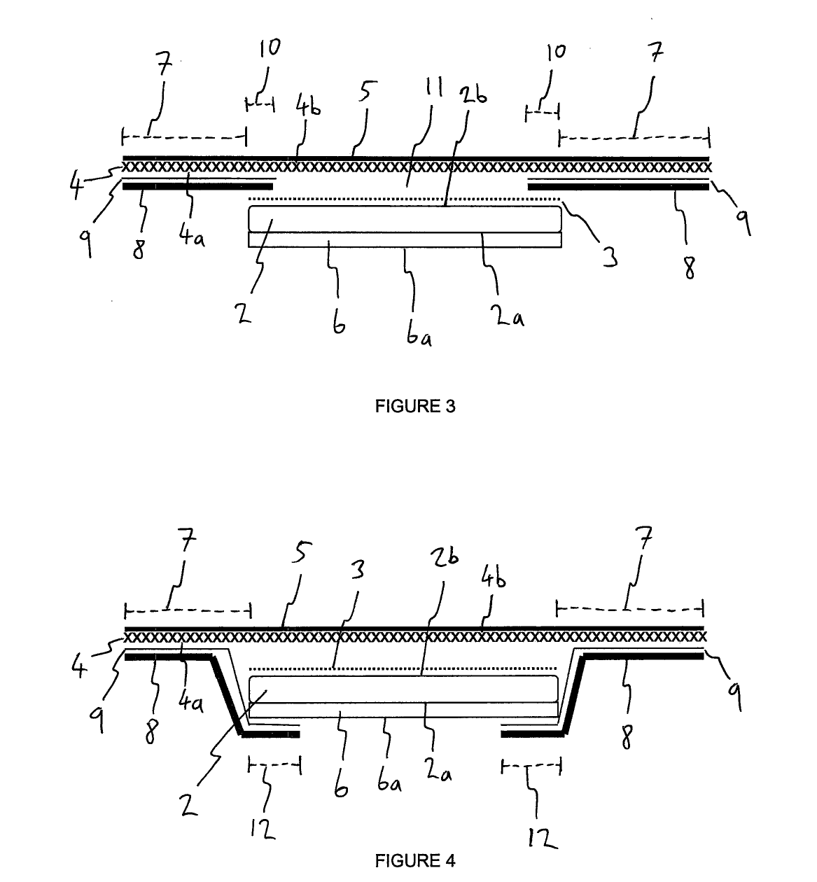

FIG. 3: is a cross-sectional representation of a wound dressing composition of the present invention comprising a skin protection layer;

FIG. 4: is a cross-sectional representation of a further alternative wound dressing composition of the present invention comprising a skin protection layer;

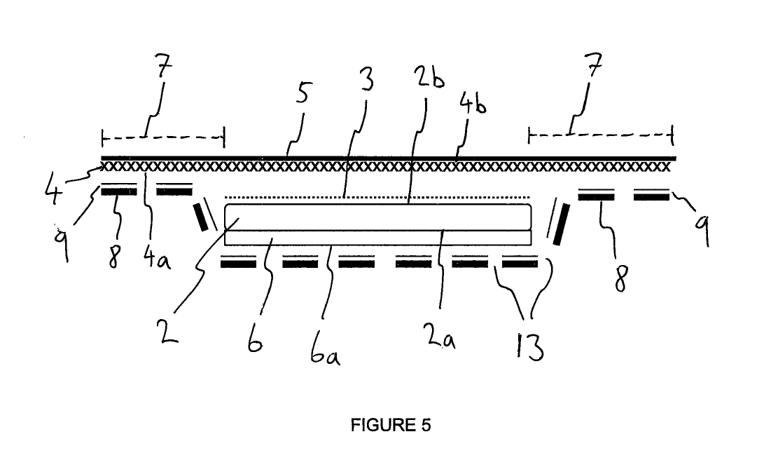

FIG. 5: is a cross-sectional representation of a further alternative wound dressing composition of the present invention comprising a skin protection layer;

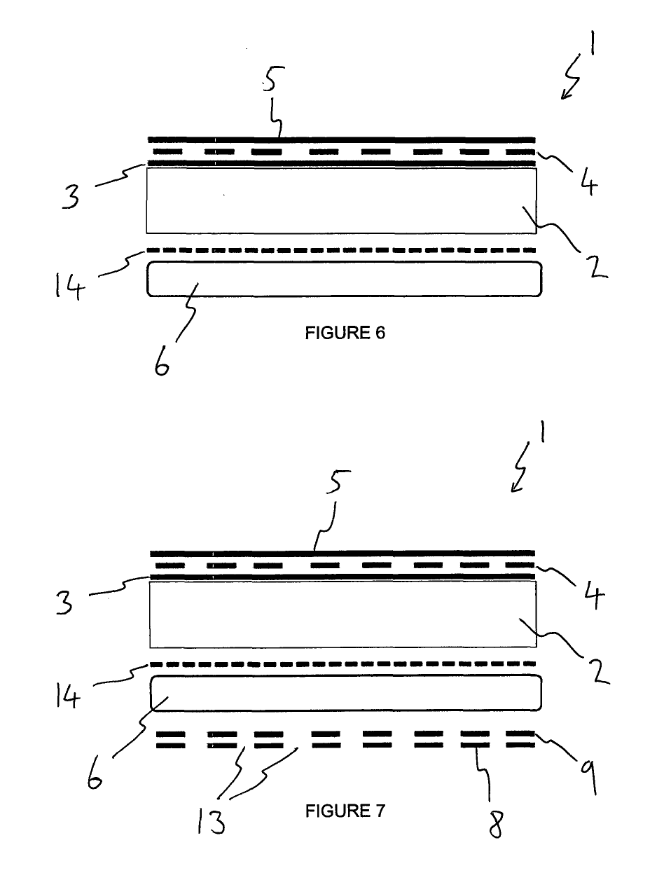

FIG. 6: is a cross-sectional representation of a further alternative wound dressing composition of the present invention;

FIG. 7: is a cross-sectional representation of the wound dressing composition of FIG. 6 further comprising a skin protection layer;

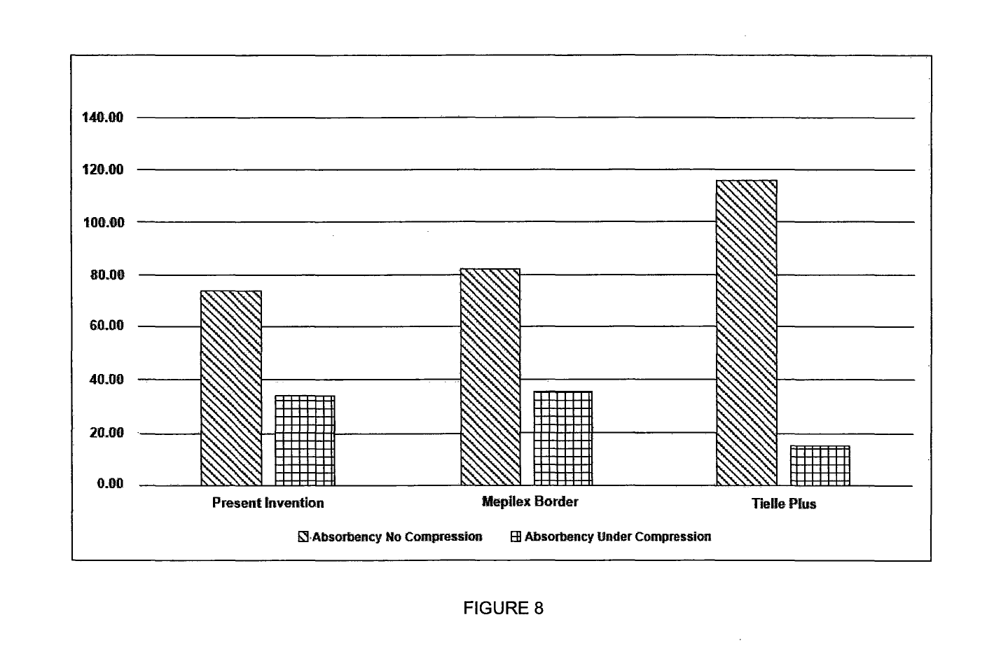

FIG. 8: is a graph showing the absorbency of the three test materials with and without compression.

Referring to FIGS. 1 and 2, there is shown a wound dressing composition (1) comprising a layer of absorbent material (2), a layer of anchor material (3) a layer of adhesive material (4), a backing layer (5) and a wound contact material (6).

The wound contact material (6) is adjacent to the wound site and will come into direct contact with the wound upon application of the wound dressing composition (1) to a wound. The absorbent material (2) has a wound facing surface (2a) and a non-wound facing surface (2b). The wound contact material (6) is attached to the wound facing surface (2a) of the absorbent material (2) by any of the means described herein. Preferably, the wound contact material (6) is attached to the absorbent material (2) using a powder adhesive. The wound contact layer (6) also serves to prevent or reduce the leaching of material from the absorbent layer (2).

The anchor material (3) is attached to the non-wound facing surface (2b) of the absorbent material (2). Typically, the anchor material (3) is heat-bonded to the absorbent material (2). As described herein, the bond created between the anchor material (3) and the absorbent material (2) is such that it will not break when the respective materials get wet with wound fluid during use.

The anchor material (3) is attached to the wound facing surface (4a) of the adhesive material (4) by any of the means described herein. Typically, the adhesive material (4) is a pressure sensitive adhesive. Thus, the anchor material (3) is contacted with the wound facing surface (4a) of the adhesive material (4) and pressure is applied to bring the two materials together.

The adhesive layer (4) has a backing layer (5) attached to its non-wound facing surface (4b). As with the anchor material (3), the backing layer (5) can be attached to the backing layer (5) by contacting the two materials together and applying pressure.

As can be seen in both FIGS. 1 and 2, the adhesive layer (4) and the backing layer (5) have a greater cross-sectional area than the anchor material (3), the absorbent material (2) and the wound contact material (6), creating a border portion (7). The wound facing surface (4a) of the adhesive layer (4) in the border portion (7) is, in use, applied directly to the patient's skin surrounding the wound site. Thus, the adhesive layer (4) has the dual purpose of adhering to the anchor layer (3) and the skin of the patient. Again, the border portion (7) of the adhesive layer (4) can be attached to the skin by applying downward pressure to the border portion (7) of the backing layer (5).

Turning to FIG. 2, the adhesive layer (4) provides less than 100% coverage of the anchor layer (3) or the backing layer (5). In this embodiment, the adhesive layer (4) is made up of regular intervals of adhesive to which the backing layer (5) and anchor layer (3) bind. This has benefits in that there is less adhesive in direct contact with the patient's skin, which will reduce discomfort upon removal of the wound dressing composition (1).

Referring to FIGS. 3, 4 and 5, there is shown wound dressing compositions (1) comprising a layer of absorbent material (2), a layer of anchor material (3) a layer of adhesive material (4), a backing layer (5), a layer of wound contact material (6) and a silicone layer (8) on a carrier material (9).

In FIG. 3, the silicone layer (8) is attached to a carrier layer (9) which is in turn attached to the wound facing surface (4a) of the adhesive material (4). The silicone layer (8) and the carrier layer (9) extend outwardly to the outer edge of the adhesive layer (4) and backing layer (5). The silicon layer (8) and the carrier layer (9) both extend inwardly to cover the whole of the border portion (7) and create an overlap (10) with the anchor layer (3). An aperture (11) is provided through which the anchor layer (3) can bind to the wound facing surface (4a) of the adhesive layer (4). The overlap (10) increases the strength and stability of the wound dressing composition (1) as the adhesive layer (4) is attached to both the anchor layer (3) and the silicone layer (8). In use, the silicone layer (8) in the border portion (7) will adhere to the skin surrounding the wound site. Beneficially, the silicone layer (8) can be removed and reapplied with less irritation and pain than the adhesive layer (4).

In FIG. 4, the silicone layer (8) extends from the edge of the adhesive layer (4), across the border portion (7) and overlaps (12) with the wound facing surface (6a) of the wound contact material (6). The overlap (12) increases the strength and stability of the wound dressing composition (1) as the silicone layer (8) more securely holds the anchor layer (3), the absorbent layer (2) and the wound contact layer (6) in place. Further, the silicone layer (8) will provide a gentle adhesion to the wound site in the area of overlap (12).

In FIG. 5, the silicone layer (8) extends from the edge of the adhesive layer (4), over the border portion (7) and across the whole of the wound facing surface (6a) of the wound contact layer (6). The silicone layer (8) and the carrier layer (9) contain regular perforations (13). The perforations (13) improve the breathability of the wound dressing composition (1) and also allow wound fluid to flow through to the wound contact layer (6). A benefit to the silicone layer (8) covering the wound facing surface (6a) of the wound contact layer (6) is that it will further prevent the leaching into the wound of any material from the wound contact layer (6) and/or the absorbent layer (2).

In FIG. 6, the absorbent layer (2) extends to the edge of the dressing (1), as does the anchor layer (3), the adhesive layer (4) and the wound contact layer (6). FIG. 6 also shows the presence of a powder adhesive layer (14) which attaches the absorbent material (2) to the wound contact layer (6).

FIG. 7 shows the wound dressing of FIG. 6, further comprising a skin protection silicone layer (8) on a carrier layer (9). The silicone layer (8) and the carrier layer (9) extend to the edge of the dressing (1).

The embodiment shown in FIGS. 6 and 7 is beneficial over the prior art, since the absorbent material (2) can extend to the edge of the dressing (1) and does not need to be encapsulated by an encapsulating material to secure it in place during use. Existing dressings require the absorbent pad to be encapsulated within the dressing area such that on absorption of fluid it does not delaminate from the dressing structure. This is achieved either by encapsulating the absorbent material within an encapsulating material such that the absorbent material is restricted to being smaller in size than the encapsulating material or incorporating an all over wound contact layer that encapsulates the absorbent pad area between the wound contact layer and the outer layer.

In use, the wound dressing composition (1) of the present invention is applied to a wound by contacting the wound contact layer (6) and/or the silicone layer (8) with the wound site. The wound dressing composition (1) can be affixed to the patient's skin by applying downward pressure to the border portion (7). Wound exudates from the wound will be absorbed by the wound contact layer (6) and drawn through to the absorbent layer (2). This has the effect of drawing fluid away from the wound bed, creating a moisture level at the wound bed that is more conducive to healing.

### EXAMPLES

The wound dressing compositions of the present invention do not delaminate when the composition is exposed to fluid, such as wound exudate. To test this, the following experiment was followed.

Test Methodology

An island wound dressing as shown in FIG. 1 was prepared. The wound dressing was made up of a superabsorbent layer (2) of polyacrylate fibres gsm 250, an anchor layer (3) of knitted fibrous polyethylene, an adhesive layer (4) of a pressure-sensitive acrylic adhesive 20 gsm, a backing layer (5) of a polyurethane film, 15 micron thickness and a wound contact layer (6) of a polyurethane foam, 1.5 mm thickness.

Three 50 mm×50 mm section of the island wound dressing were cut out and immersed for a minimum of 24 hours in three separate solutions A, B and C. For comparison purposes, prior art dressings Mepilex Border (Molnlycke Healthcare) and Tielle Plus (Systergenics) were tested. On submersion in the below test fluids, the Mepilex Border and Tielle dressings observed that the backing layers separated from the absorbent layers. This was not observed for the wound dressing of the present invention. When tested as a whole dressing, only the Tielle exhibited separation of the backing material from the absorbent pad materials.

In the tests, Solution A is 142 mmol sodium ions and 2.5 mmol calcium ions as the chloride salt, Solution B is saline and Solution C is simulated wound fluid (50% peptone water and 50% fetal bovine serum).

The three sections of wound dressing were then inspected for integrity.

Finally, the tensile strength of the superabsorbent layer bonded to the acrylic adhesive layer was measured using a tensiometer (>0.5N/25 mm). For the Mepilex Border and Tielle Plus dressings, no results were obtained as the layers had separated. The material of the present invention achieved a range of from 0.65N/25 mm to 1.1N/25 mm.

An island wound dressing as shown in FIG. 3 was prepared and tested by the same test methodology described above. The silicone layer (8) was attached to a polyurethane film layer (9) which was attached to the wound facing surface (4a) of the adhesive layer (4). The polyurethane layer (9) is the same as the polyurethane backing layer (5).

The test further included an assessment for any leaching of the superabsorbent layer (2) from the wound dressing composition (1).

No visual leaching of superabsorbent layer for any of the dressings tested.

Absorbency was also tested and the results are shown in the graph of FIG. 8.

It is clear from FIG. 8 that the dressing of the present invention shows good absorbency under compression compared to the comparative dressings Mepilex Border and Tielle Plus.

It is of course to be understood that the present invention is not intended to be restricted to the foregoing examples which are described by way of example only.

1. A wound dressing composition comprising an absorbent material, an adhesive material and an anchor material attached to the absorbent material and the adhesive material, whereby the anchor material is operable to maintain a link between the absorbent material and the adhesive material when the wound dressing is wet.

2. A composition according to claim 1, wherein the absorbent material is a superabsorbent material.

3. A composition according to claim 2, wherein the superabsorbent material comprises a polymeric material.

4. A composition according to claim 3, wherein the polymeric material is selected from PVA, PEO and polyacrylic acid.

5. A composition according to claim 1, wherein the absorbent material is in the form or fibres.

6. A composition according to claim 5, wherein the fibres form a non-woven layer.

7. A composition according to claim 1, wherein the adhesive material comprises a pressure-sensitive adhesive.

8. A composition according to claim 1, wherein adhesive material is selected from acrylic adhesives and polyurethane adhesives.

9. A composition according to claim 1, wherein the adhesive material covers from around 50-100% of a non-wound facing surface of the anchor material.

10. A composition according to claim 1, wherein the adhesive material provides a border that extends beyond one or more edges of the anchor material and the absorbent material.

11. A composition according to claim 1, wherein the anchor material is a nonabsorbent material that is not water soluble and/or water swellable.

12. A composition according to claim 1, wherein the anchor material is in the form of fibres.

13. A composition according to claim 1, wherein the anchor material comprises polyethylene.

14. A composition according to claim 1, wherein the anchor material is heat-bonded to the absorbent material.

15. A composition according to claim 1, further comprising a backing material, skin contact adhesive and/or wound contact material.

16. A composition according to claim 15, wherein the backing material comprises a polyurethane film.

17. A composition according to claim 15, wherein the wound contact material is hydrophilic.

18. A composition according to claim 17, wherein the wound contact material comprises polyurethane foam.

19. A composition according to claim 1, further comprising a skin protection layer attached to a wound facing surface of the adhesive material.

20. A composition according to claim 19, wherein the skin protection layer comprises silicone.

21. A composition according to claim 19, further comprising a carrier layer located between the skin protection layer and the adhesive material.

22. A composition according to claim 21, wherein the carrier layer comprises a polyurethane film.

23. A composition according to claim 19, wherein the skin protection layer overlaps with a non-wound facing surface of the anchor material, or the skin protection layer overlaps with a part of the wound facing surface of the absorbent material or, if present, the wound facing surface of the wound contact material.

24. A composition according to claim 23, wherein the skin protection layer extends across all of the wound facing surface of the absorbent material or, if present, the wound contact material.

25. A composition according to claim 24, wherein the skin protection layer is perforated.

26. A wound dressing comprising a wound dressing composition according to claim 1.

27. A method of manufacturing a wound dressing composition as described herein, comprising the steps of:
(a) providing an absorbent material,
(b) attaching the absorbent material to a surface of an anchor material,
(c) attaching the opposing surface of the anchor material to an adhesive material,
whereby the anchor material is operable to maintain the link between the absorbent material and the adhesive material when the wound composition is wet.

28. A method according to claim 27, wherein the absorbent material is attached to the anchor material by heat-bonding.

29. A use of a wound dressing composition according to claim 1, in absorbing fluid discharged from a physiological target, or in stemming a flow of a fluid discharged from a physiological target site.

30. A wound dressing composition according to claim 1, for use in absorbing fluid discharged from a physiological target, or for use in stemming a flow of a fluid discharged from a physiological target site.

31. (canceled)
